(19) United States
(12) Patent Application Publication
(10) Pub. No.: US 2004/0248204 A1
(43) Pub. Date: Dec. 9, 2004

(54) Method and device for utilizing analyte levels to assist in the treatment of diabetes, insulin resistance and metabolic syndrome

(76) Inventor: Piet Moerman, St. Martens-Latem (BE)

Correspondence Address: LAHIVE & COCKFIELD, LLP., 28 STATE STREET, BOSTON, MA, 02109, US

(21) Appl. No.: 10/817,211

(22) Filed: Apr. 1, 2004

Related U.S. Application Data
(60) Provisional application No. 60/459,310, filed on Apr. 1, 2003.

(51) Int. Cl. A61K 38/28; G01N 33/53; G06F 19/00; G01N 33/48; G01N 33/50

(52) U.S. Cl. 435/7.1; 702/20; 514/3

(57) ABSTRACT

A health-monitoring device assesses the health of a user based on levels of two analytes in a biological fluid. A first analyte that is utilized to assess a user's health is a fat metabolism analyte, such as ketones, free fatty acids and glycerol, which is indicative of fat metabolism. A first analyte that is utilized is a glucose metabolism analyte, such as glucose. The levels of the two analytes may be used to assess insulin sensitivity, to detect both recent hypoglycemia and the cause of high glucose levels, and/or to guide therapeutic intervention. The dual analyte model of the present invention may be used to identify individuals at risk for metabolic syndrome, insulin resistance and non-insulin dependent diabetes, and allows monitoring of the progression of those disease states, as well as progress made by therapeutic interventions.

### RELATED APPLICATIONS

The present invention claims priority to U.S. Provisional Patent Application 60/459,310 entitled Method and Device for Utilizing Analyte Levels to Assist in the Treatment of Diabetes, Insulin Resistance and Metabolic Syndrome, filed Apr. 1, 2003, the contents of which are herein incorporated by reference.

### FIELD OF THE INVENTION

The present invention relates to the management of metabolic syndromes, diabetes and cardiovascular risk. More particularly, the present invention relates to systems and methods for managing metabolic syndrome, diabetes and cardiovascular risk using quantification of biochemical markers in the subject to assess the fat and glucose metabolism and insulin sensitivity.

### BACKGROUND OF THE INVENTION

Between 1990 and 1998 the prevalence of diabetes in the United States rose from 4.9 to 6.5%. During the 1990's the prevalence of non-insulin dependent diabetes increased by 33% overall and by 70% among people in their thirties. Diabetes affects now sixteen million Americans. The direct costs resulting from diabetes is $44 billion per year, and the total cost of diabetes, including indirect costs, rises to $98 billion per year. 13.5% of obese patients have diabetes compared to 3.5% of those with a normal weight.

Diabetes is the “tip of the Iceberg” and is most often preceded by a metabolic syndrome. The prevalence of the metabolic syndrome gives an estimate of the potential magnitude of the problem. The Centers of Disease Control and Prevention recently investigated the prevalence of the metabolic syndrome: The unadjusted and age-adjusted prevalences were 21.8% and 23.7%, respectively. The prevalence increased from 6.7% among participants aged 20 through 29 years to 43.5% and 42.0% for participants aged 60 through 69 years and aged at least 70 years, respectively. Using 2000 census data, about 47 million US residents have the metabolic syndrome.

The main concern is that those metabolic syndrome patients are cardiovascular compromised and evolve spontaneously from insulin resistance, to diabetes and cardiovascular incidents.

### SUMMARY OF THE INVENTION

The present invention provides a comprehensive approach to the management of metabolic syndrome, insulin resistance and diabetes. The method of the present invention utilizes dual parameters in understanding metabolic changes in the body. A first parameter that may be utilized in accordance with the present invention may comprise biochemical signals indicative of fat metabolism (e.g., Ketones or Free Fatty Acids or Glycerol levels) and a second parameter may comprise biochemical signals indicative of glucose metabolism (e.g., glucose levels). These measurable signals, in blood or other bodily fluids, may be used to assess insulin sensitivity, to detect both recent hypoglycemia and the cause of high glucose levels, and/or to guide therapeutic intervention. The dual analyte model of the present invention may be used to identify individuals at risk for metabolic syndrome, insulin resistance and non-insulin dependent diabetes. Furthermore, the dual analyte model allows monitoring of the progression of those disease states, as well as progress made by therapeutic interventions. For insulin dependent diabetes in particular, the dual analyte model can help in the dosing of medication (insulin and others) and of dietary changes.

The present invention provides a single device for testing both a fat metabolism analyte and a glucose metabolism analyte, as well as for interpreting the combined results of the dual analyte measurements.

According to a first aspect of the invention, a method of assessing a user's health comprises the computer implemented steps of measuring an amount of a first analyte in a biological fluid sample reflecting body fat metabolism and an amount of a second analyte in the biological fluid sample reflecting glucose metabolism, and assessing the health of the user based on the amount of the first analyte and the amount of the second analyte.

According to another aspect of the invention, a health-monitoring device for assessing a user's health comprises a sampling device for providing a biological fluid sample from the user and one or more test elements. The test elements measure an amount of a first analyte in the biological fluid sample reflecting body fat metabolism and an amount of a second analyte in the biological fluid sample reflecting glucose metabolism.

According to still another aspect of the invention, a method for monitoring the health of a user comprises the steps of measuring a level of a first analyte reflecting body fat metabolism in a biological fluid sample, determining a glucose level in a biological fluid sample and calculating and tracking the evolution of an insulin resistance factor in the user based on the measured level of the first analyte and the glucose level.

In yet another aspect of the invention, a method of monitoring a health parameter in a user comprises the computer implemented steps of measuring an amount of a fat metabolism analyte in a biological fluid sample reflecting body fat metabolism, measuring an amount of a glucose metabolism analyte in the biological fluid sample, and correlating the amount of the fat metabolism analyte and the amount of the glucose metabolism analyte to the health parameter.

### BRIEF DESCRIPTION OF THE DRAWINGS

FIGS. 1a and 1b illustrate an electronic health monitoring device for sampling and analyzing a biological fluid sample and assessing the health of a user based on levels of two analytes in the sample.

FIG. 2 illustrates the output and user interface of the device of FIGS. 1a and 1b when tracking an insulin resistance factor.

FIG. 3a is a schematic of a health monitoring system including the health monitoring device of FIGS. 1a and 1b.

FIG. 3b is a block diagram showing the components of the processor of FIG. 3a.

FIG. 4 shows the display of the device of FIGS. 1a and 1b when the device is used to track an intra-day evolution of glucose and FFA levels and display a warning about imminent hypoglycemia, according to an embodiment of the invention.

FIG. 5 shows the display of the device of FIGS. 1a and 1b when the device is used to display early morning test results for glucose and FFA and the interpretation thereof, according to an embodiment of the invention.

### DETAILED DESCRIPTION OF THE INVENTION

The present invention provides a system and method for managing diabetes, insulin resistance, metabolic syndrome and obesity. The system and method of the present invention tracks dual parameters and utilizes the dual parameters to understand metabolic changes and provide therapeutic advice to a user. The invention will be described below relative to illustrative embodiments. Those skilled in the art will appreciate that the present invention may be implemented in a number of different applications and embodiments and is not specifically limited in its application to the particular embodiments depicted herein.

As used herein, the terms “fat analyte” and “fat metabolism analyte” refer to an analyte generated in a patient when consuming body fat. Fat analytes and fat metabolism analytes include, but are not limited to, ketones, glycerol, Free Fatty Acids (FFA) and a fatty acid that is representative of the total FFA's in the system, such as Palmitate. Free Fatty Acids are a family of different fatty acids, and traditional test systems for Free Fatty Acids measure the most representative fatty acid of the family, which is usually Palmitate. However, one skilled in the art will recognize that other fatty acids present in other proportions are also representative of a total FFA level and may also be used. In particular, long chain saturated Fatty Acids, such as, but not limited to stearate, arachidate and others, may be of interest as they have shown to have particular delirious effects on the metabolism.

As used herein, the terms “glucose analyte” and “glucose metabolism analyte” refer to an analyte indicative of glucose metabolism. Metabolic analytes indicative of glucose metabolism include, but are not limited to, glucose levels, pyruvate, glucose6phosphate and lactate.

The term “biological fluid” as used herein refers to a fluid containing a metabolic analyte, including, but not limited to blood, derivatives of bloods, interstitial fluid, urine, a breath sample, saliva, and combinations thereof.

As used herein, the term “health parameter” is intended to include any parameter associated with correlated with, or indicative of the health of the user. Examples a health parameter include, but are not limited to, an insulin sensitivity factor, a medication dosage, an insulin dosage, an assessment of a metabolic syndrome, a likelihood of the user developing hypoglycemia or hyperglycemia and a likelihood that the user recently developed hypoglycemia.

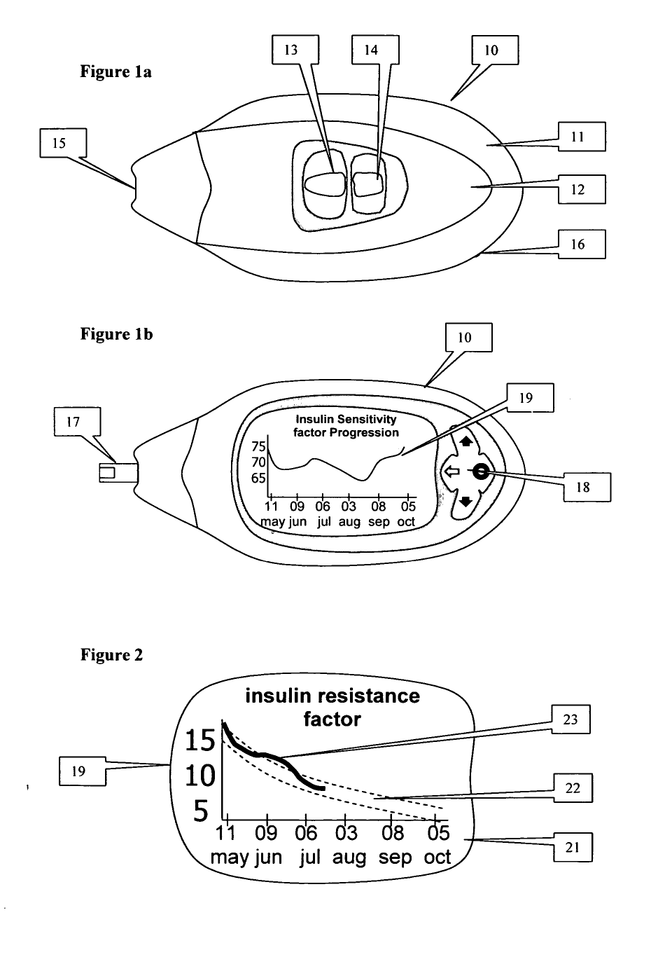

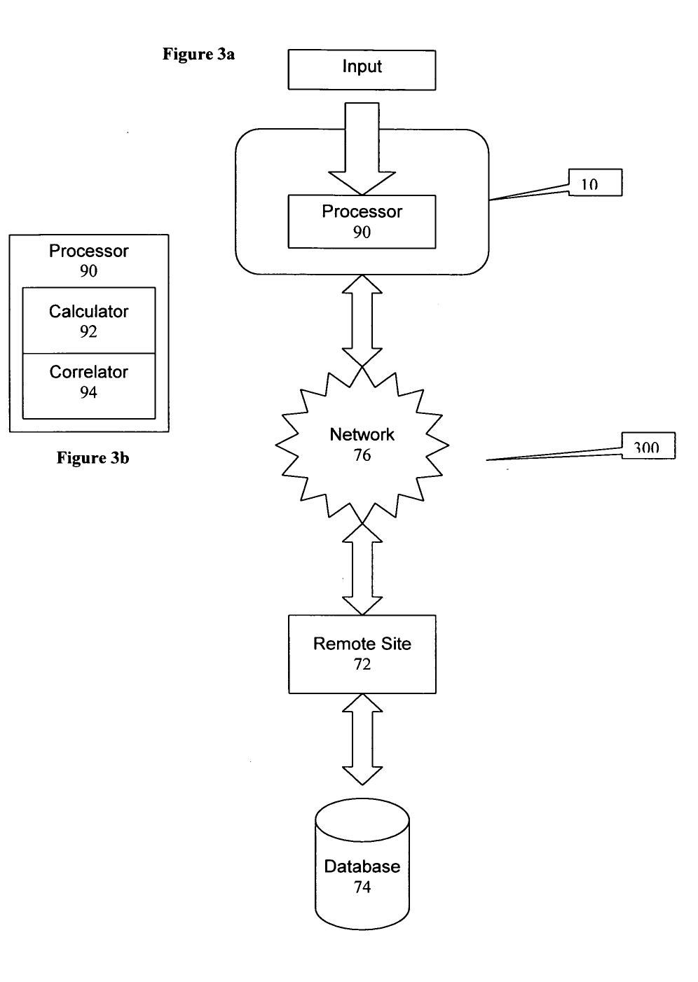

FIGS. 1a, 1b, 2, 3a, and 3b, illustrate a health-monitoring device or monitor 10 for monitoring the health of a patient according to an illustrative embodiment of the invention. The illustrative health-monitoring device 10 includes a sampling device for sampling a biological fluid, such as blood, and a testing device for measuring the levels of two analytes in the sample, for example a fat analyte and a glucose analyte, through means known in the art. The device 10 includes a processor 90, which is shown in FIGS. 3a and 3b, for running a program that uses the measured analyte levels to assess the health of a user. In one embodiment, the device 10 correlates fat analyte and glucose analyte measurements to a health parameter to give the user an assessment of his health. The health-monitoring device includes a display 19 for displaying results to the user, as well for providing the different options in tracking results and reading the advice. For example, as shown in FIG. 2, the illustrative device 10 calculates an insulin sensitivity factor based on the measured levels of two analytes in a user. As shown, the health-monitoring device 10 tracks the progress of the user's insulin sensitivity factor over time to provide feedback to the user regarding his health.

According to the illustrative embodiment, the health monitoring device 10 measures a fat analyte, which is indicative of fat metabolism in the user, and a glucose analyte, which is indicative of glucose metabolism in the biological fluid sample and uses the two measurements to calculate a health parameter. The fat analyte may comprise free fatty acids (FFA), ketones, glycerol or any other analyte that is indicative of lipolysis (fat breakdown) in the body.

As shown, the device 10 includes a housing 11, which incorporates a sampling device, illustrated as a lancing device 12 having a lancet, for piercing the skin of a user. The sampling device is used to yield a biological fluid sample containing one or more of the analytes to be measured. The lancing device 12 may include a variable depth selector 14 for setting the penetration depth of the lancet and a trigger button 13 for releasing the lancet to prick the skin. One skilled in the art will appreciate that the lancing device does not have to be incorporated into the health-monitoring device 10 but can be a separate stand alone device. Alternatively to the lancet, a hollow needle may be used to extract the sample from or from within the skin. The sampling device may comprise any suitable means for yielding a biological fluid sample and is not limited to a lancing device or other device for piercing the skin of a user.

The illustrative testing device 10 includes a test port 15, which allows a disposable test element 17 to be inserted into the apparatus. The test element 17 may comprises any suitable device for measuring analytes, including, but not limited to a test strip, a skin inserted device, such as a catheter, or a measuring device that uses a non-invasive methods of measurement which may not utilize a body fluid sample. The test element 17 generates a signal indicative of the concentration of the tested metabolic analytes in the sample, which can be based either on a photometric, electrochemical analytical method or any other suitable method known in the art. The test port 15 may include electrical contacts for reading the signal of an electrochemical based test strip or may hold a photometric or reflectometric cell to read the signal of a photometric test strip. Other readers can be used in accordance with the teachings of the invention, including, but not limited to a fluorescence reader, magnetic reader, and others known to those of ordinary skill in the art, depending on the utilized test element or assay technology.

One single test element 17 may be utilized to measure both analytes, so that the patient has to sample only once (i.e. stick his finger to obtain a blood drop) to obtain both results. Alternatively, a different test element can be used for each analyte measured in the patient.

Based on the measured levels of the analytes in the biological sample, a processor in the health-monitoring device 10 calculates a health parameter and provides feedback to the user regarding the calculated health parameter.

A data communication port 16 in the housing 11 allows insertion of an electrical connector to access the electronics in the device 10. This feature can be used to download, as well as upload, data and programs. One skilled in the art will recognize that communication between the electronics is not limited to electrical communication. Acoustic, optic (infrared), radio waves or other communication means known in the art may be used as well.

The illustrative device 10 may include an interface button 18 for navigating menu options presented on the display 19 or to select and confirm data inputs and outputs.

The correlation between the measured analyte levels and the health of a user, assessed using a program stored in the device 10 of FIGS. 1a and 1b, will be described in greater detail below.

The illustrated monitor 10 contains electronics, including a processor 90 for reading and receiving a signal from the test element 17, shown in FIGS. 3a and 3b. By using the calibration information for the test element, the processor 90 can convert the measured signals generated by the test element 17 to a concentration of each of the tested metabolic analytes. The processor 90 provides feedback to a user based on the levels of the first and second metabolic analyte in a biological fluid sample. The processor 90 includes a calculator 92 for determining the level of the first metabolic analyte, such as a fat analyte, and a second metabolic analyte, such as a glucose analyte in the sample. The processor 90 also includes a correlator 94 for correlating the levels of the first and second metabolic analyte to a health parameter indicative of the user's health. The measured analyte concentration can be displayed on the display 19 and/or stored into memory of the monitor 10.

In one embodiment, as shown in FIG. 3a, the monitor 10 may form part of a health monitoring system 300. The health monitoring system comprises the monitor 10 and a remote site 72 having a database 74 for storing data obtained by the monitor 10. As shown, the monitor may be connected to the remote site 72 over a network 76.

According to an illustrative embodiment of the invention, the health monitoring device 10 utilizes and implements relationships between fat analytes and glucose analytes in the body and parameters indicative of the health of a user. The processor 90 may be programmed to calculate a health parameter based on known relationships between levels of fat analytes and glucose analytes and certain health parameters. For example, in the human body, levels of free fatty acids (FFA) rise when there is a rise in insulin action and a raise in counter-regulating hormones. Obesity is also commonly associated with elevated plasma free fatty acid (FFA) levels, as well as with insulin resistance and hyperinsulinemia, two important cardiovascular risk factors.

Free Fatty Acids and Lipid Metabolism

A drop in insulin action and a rise in counter-regulating hormones also tends to cause a rise in Free Fatty Acids (FFA) in the human body. Adipose tissue plays an important role in energy supply. In the absence of sufficient glucose to meet the body's energy needs, lipolysis, i.e., fat breakdown, supplies Free Fatty Acids for energy. Body fat is broken down to release Free Fatty Acids (FFA) and glycerol into the circulation. This typically occurs in the post-absorptive phase (the time span between the digestion of a meal and the start of the next meal) and overnight (the longest fasting period of the day). The regulation of lipolysis is under control of a variety of hormones, including insulin, glucagon, growth hormone (GH), epinephrine, adrenalin and cortisol.

Under caloric restriction, glucose levels in the body drop progressively, and then stabilize. As a reaction, plasma levels of insulin drop while glucagon levels increase. The result of this decreased insulin/glucagon ratio is a lipolytic effect on the fat tissue, which releases FFA into the blood stream. The FFA generally have two destinations: some are consumed directly by the body tissues for energy, other enter the liver cells for ketogenesis (beta-oxidation to form ketones). In addition, glucagon will also stimulate the liberation of glucose from the liver and muscle stores to compensate for a shortage of glucose.

Besides glucagon, other hormones will try to compensate for the shortage of glucose. Growth hormone, for example, plays an important role during the night to ensure sufficient energy substrates are available. Growth hormone (GH) infusion in normal subjects increases glycerol and FFA concentrations, indicating an enhanced lipolysis. The ketogenetic effect of growth hormone is explained by the increase of substrate (FFA) through enhanced lipolysis. Growth hormone secretion is typically increased early in the night to compensate for dropping glucose levels in the blood. Dropping glucose concentration and insulin levels triggers the increase in GH secretion. In insulin dependent diabetes, GH secretion is markedly increased, especially in adolescents and patients with poorly controlled diabetes.

It has been suggested that there is a negative feedback loop between FFA and Growth Hormone. Lack of FFA itself may be the signal for growth hormone release despite the lag (generally about 2 hours) period between FFA decrease and Growth hormone increase. Glucose and FFA can at least not fully replace each other in their respective influence on growth hormone.

GH effects during the night may play an important role to the origin of the “dawn-phenomenon” found in diabetic patients, a low glucose level during the night followed by a high glucose at wake with an increased need for insulin. The typical increase of FFA, most often a doubling of the baseline levels, generally occurs between 2 and 3 hours after the GH peak.

Adrenaline and epinephrine, two hormones produced under stress conditions also stimulate lipolysis in a attempt to ensure sufficient energy substrates.

In summary, FFA rises due to a drop in insulin action and a raise in counter-regulating hormones. The raise in counter-regulating hormones is influenced by a couple triggers, which can sometimes be unpredictable, such as meal intake during the day and GH, stress and nervosa for adrenalin and epinephrine, the circulating level of insulin and the glucose concentration in the blood. Therefore, the rise in FFA's and their association with insulin sensitivity and glucose levels is unpredictable and justifies the need for frequent monitoring.

Lipolytic Parameters.

According to an illustrative embodiment, the monitoring devices measures and correlates Free Fatty Acid levels to analyze a user's health, though one skilled in the art will recognize that any analyte that reflects lipolysis can be used. For example, other analytes, such as ketones and glycerol are also products from lipolysis, and can be used to assess the effect of the counter-regulating hormones in the body. As described above, under a condition of low insulin, low glucose and high counter-regulating hormones (such as, but not limited to Growth hormone, glucagon, cortisol, epinephrine and noradrenaline), lipolysis is stimulated to supply other sources of energy than glucose. Body fat is stored as triglycerides, which is a molecule made up of three free fatty acid (FFA) chains and one glycerol. Lipolysis will thus liberate FFA and glycerol from the fat stores into the circulation. The FFA's can enter body cells (but not neural tissue cells), and be oxidized. FFA can also enter the liver mitochondria and be converted to ketones, also a source of energy but in high concentration those can be toxic. Glycerol will contribute to the new formation of glucose.

Dieting and Lipolytic Parameters

People on hypo-caloric diets usually have higher pre-prandial Free Fatty Acid levels. This is due a compensatory mechanism where the energy needs are mainly met by body fat breakdown. Non-insulin dependent patients on calorie restriction may see similar events, where higher levels of FFA's are measured before the meal compared with a normal iso-caloric diet. Weight loss is associated with a reduction of body fat, the supply source of FFA's. Generally, base levels (i.e., after the meal) of FFA will drop as the patient loses weight, associated with restoring his insulin sensitivity. Therefore, for people on a calorie restricted diet, post-prandial FFA levels or certain differences in FFA levels may be measured to assess an insulin sensitivity factor using the health-monitoring device 10 of an illustrative embodiment of the invention.

Cardiac Risk and Free Fatty Acids

The obese, metabolic syndrome and non-insulin dependent patient has a typical dyslipidemia (high triglycerides, low HDL-cholesterol, increased small dense LDL Cholesterol) to a larger extent caused by high levels of FFA's. As extensively documented, this dyslipidemia is a major risk factor in the development of cardiovascular disease. About 70-80% of non-insulin dependent patients will die from a macro vascular complication of their disease, for example, myocardial infarction, hearth failure, stroke, aneurysm. While micro vascular complications (blindness, kidney failure, neuropathy, skin lesions) are caused by high glucose levels, the macro vascular complications are indirectly caused by high FFA levels. It has been suggested that Nitric Oxide (a vasodilatator) production in response to different stimuli may be mediated via different signaling pathways. FFA-induced reduction of NO production may contribute to the higher incidence of hypertension and macro-vascular disease in insulin-resistant patients. (See “Free Fatty Acid Elevation Impairs Insulin-Mediated Vasodilatation and Nitric Acid Production” Diabetes, 2000;49:1231-38.) An oversupply of FFA from visceral fat at the liver, stimulates the production of Triglycerides and VLDL, ultimately resulting in low levels of HDL-Cholesterol and high levels of LDL-Cholesterol. This is the typical dyslipedemia associated with an increased mortality due to cardio-vascular incidents. In addition to the insulin resistance information, monitoring FFA levels is important to help reduce the subject's risk for cardiovascular incidents or death.

Non-Insulin Dependent Diabetes

Non-insulin dependent diabetes is characterized by an insufficient insulin action, which typically begins with the development of insulin resistance in the obese. Obesity, and consequent insulin resistance, may be present and deteriorating years before a glucose tolerance test for detecting diabetes is capable of detecting diabetes symptoms. For example, insulin resistance can be detected in patients up to 10 years before manifestation of the typical non-insulin dependent diabetes symptoms or diagnosis of diabetes.

The pancreas, through an increase in insulin secretion, initially compensates insulin resistance. A patient's pancreas can cope for years with the increasing insulin resistance and maintain normal glucose levels and glucose tolerance tests. Insulin production can raise a threefold. However, at a certain point, the need for insulin exceeds the capacity of the defaulting B-cells in the pancreas and insulin production becomes insufficient to cope with the increasing insulin resistance. At this stage, the patient starts to develop impaired glucose tolerance test results. While the disease continues to develop, the muscles, which are the least insulin sensitive organ, become unable to extract glucose from the bloodstream. This phenomenon typically occurs after consuming a meal (rich in carbohydrates), and results in post-prandial hyperglycemia.

At a further stage, the insulin action may become so impaired that the liver cannot fully function either. Under normal circumstances, the liver extracts glucose from the bloodstream and stores the extracted glucose as glycogen when high levels of insulin are present. When glucose levels drop, the insulin level may drop as well. The resulting drop of insulin and the increase of glucagon cause the liver to breakdown its glycogen reserves and release glucose into the blood stream. This glucose homeostasis is essential as an energy source for the brain and neural tissue. When insulin resistance worsens and the production of insulin drops, the liver function will be affected. The liver becomes less stimulated by insulin and tends to release glucose at a higher level. In addition, glucose utilization by the peripheral tissues becomes diminished due to the reduced insulin action. This results in high glucose levels overnight and at fasting (pre-breakfast hyperglycemia). Ultimately, the B-cells from the pancreas are exhausted and insulin levels start to drop, resulting in full blown diabetes disease.

Non-Insulin Dependent Diabetes, Obesity and Free Fatty Acids

There is evidence of a relationship between insulin resistance and body fat, which is exploited using the health-monitoring device 10 of the present invention. For example, it is estimated that about 83% of all diabetes patients are overweight, while 13.5% of obese patients have diabetes compared to 3.5% of those with a normal weight. Weight control intervention is desired and proven to be a most effective first step in the therapy of most non-insulin dependent diabetics.

In non-insulin dependent diabetes and insulin resistant patients, obesity typically increases lipid oxidation. The preferential use of FFA for energy supply is responsible for the decrease in glucose mobilization from glycogen stores. This leads through a negative feedback of muscle and liver glycogen on glycogen synthetase activity and consequently in the reduced extraction of glucose from the bloodstream. The reduced extraction of glucose from the bloodstream results in high levels of glucose after carbohydrate ingestion, which is related to the competition for the citric acid cycle by both the FFA and glucose. Due to the large adipose tissue reserves, more FFA are released into the circulation and stiffen the competition with glucose for the citric acid cycle. Diabetes develops in obesity, usually after a long period of glucose intolerance, where glycemia does not return to the basal state. In obesity, glucose intolerance and insulin resistance can be prevented, or if already existing, can be decreased by stimulating glycogen mobilization by exercise and weight loss, which reduces fat stores and decreases lipid oxidation.

There is significant evidence emerging that the FFAs in the blood stream may be the cause of the insulin resistance. Normal people on a low-carbohydrate diet and diabetics both show antagonism, not only to hypoglycemic action of insulin, but also to the important action of insulin in suppressing release of FFA. The abnormalities of carbohydrate metabolism (including insulin resistance) which occur in these two conditions are attributed to the release of more FFA for oxidation. The new possibility is suggested that the primary event in the development of diabetes could be an abnormality of gluceride metabolism, which leads to release of more FFA in adipose tissue and muscle. (See C. Hales, P. Randle. “Effects of Low-Carbohydrate Diet and Diabetes Mellitus on Plasma Concentrations of Glucose, Non-Esterified Fatty Acids, and Insulin During Oral Glucose-Tolerance Tests” The Lancet; 790-794; Apr. 13, 1963.) Fraze et al have found elevated (fasting and postprandial) FFA levels in non-obese patients with NIDDM despite the fact that insulin levels were elevated or not. (See “Ambient Plasma Free Fatty Acid Concentrations in Noninsulin-Dependent Diabetes Mellitus: Evidence for Insulin Resistance” J. Clin Endocr. And Metabol. Vol 61, No 5: 807-11. 1985.) A study examined the mechanism by which free fatty acids induce insulin resistance in human skeletal muscle. The data suggests that increased concentrations of plasma FFA induce insulin resistance in humans through inhibition of glucose transport activity.

Acute rises in FFA levels has been shown to inhibit the action of insulin on glucose uptake, glycogen synthesis and endogenous glucose production.

Insulin control of glucose output is a major mechanism by which appropriate amounts of glucose are produced to supply energy to the central nervous system, without causing long-term increases of the plasma glucose concentration. It is hypothesized that the primary route by which insulin maintains control over glucose production is indirect and is mediated by regulation of free fatty acid release from the adipocyte. Free fatty acids act as a signal as well as a metabolic substrate. They can regulate glucose utilization in muscle and apparently are important signals to the liver and the beta cells as well. The importance of portal vein FFA concentrations to the function of the liver could explain insulin resistance of the liver with central pattern obesity.

Obesity is commonly associated with elevated plasma free fatty acid (FFA) levels, as well as with insulin resistance and hyperinsulinemia, two important cardiovascular risk factors. In a study, Santomauro et al have found strong evidence that FFAs are the link between obesity and insulin resistance/hyperinsulinemia and that, lowering of chronically elevated plasma FFA levels improves insulin resistance/hyperinsulinemia and glucose tolerance in obese non-diabetic and diabetic subjects. (See A. T. Santomauro, G. Boden, M. E. Silva et al. “Overnight Lowering of Free Fatty Acids with Acipimox Improves Insulin Resistance and Glucose Tolerance in Obese Diabetic and Nondiabetic Subjects” Diabetes. 1999;1836-1841.)

A new class of anti-diabetic drugs tends to exert their action on the adipocyte lowering the liberation of FFA: rosiglitazone increases hepatic and peripheral (muscle) tissue insulin sensitivity and reduces FFA turnover despite increased total body fat mass. These results suggest that the beneficial effects of rosiglitazone on glycemic control are mediated, in part, by the drug's effect on FFA metabolism. (See Miyazaki, L. Glass, C. Triplit, M. Matsuda, K. Cusi, A. Mahankali, S. Mahankali, L. J. Mandarino, R. A. DeFronzo. “The Effect of Rosiglitazone on Glucose and Non-Esterified Fatty Acid Metabolism in Type II Diabetes Patients” Diabetologia. 2001; 44:2210-2219.)

In another study the effect of three months of rosiglitazone treatment (4 mg b.i.d.) on whole-body insulin sensitivity and in vivo peripheral adipocyte insulin sensitivity. In conclusion, these results support the hypothesis that thiazolidinediones enhance insulin sensitivity in patients with type 2 diabetes by promoting increased insulin sensitivity in peripheral adipocytes, which results in lower plasma fatty acid concentrations and a redistribution of intracellular lipid from insulin responsive organs into peripheral adipocytes. (See A. B. Mayerson, R. S. Hundal, S. Dufour, et al. “The Effects of Rosiglitazone on Insulin Sensitivity, Lipolysis, and Hepatic and Skeletal Muscle Triglyceride Content in Patients with Type 2 Diabetes Diabetes. 2002; 51:797-802.)

As described above, FFA levels in obese non-diabetic, obese diabetic and non-obese diabetics play an important role in both reflecting and controlling glucose metabolism and insulin sensitivity. Lowering FFA levels restores insulin sensitivity and vice versa.

Based on information regarding the relationship of FFA levels to certain health parameters of a user, the health-monitoring device 10 of an illustrative embodiment of the invention can utilize measured FFA levels to assess an insulin sensitivity factor of a patient and to track the progression of the insulin sensitivity factor.

FFA levels start to increase in the obese in conjunction with the development of insulin resistance. As these patients start to progress towards a positive glucose tolerance test and finally overt non-insulin dependent diabetes, FFA levels steadily increase as well, reflecting first the reduced insulin sensitivity and, at the end stage, the combination with reduced insulin production.

Referring back to FIGS. 1b and 2, the health-monitoring device 10 of an illustrative embodiment of the present invention may be used to measure and interpret the FFA level of a patient, taken at certain moments of the day and at certain intervals, as well as glucose level of the patient. Based on the measurement of the FFA level and the glucose level, the device calculates and displays a health parameter, such as an insulin sensitivity factor, as shown in FIGS. 1b and 2. The algorithm used to determine the insulin sensitivity factor may be based on the relationships described above, and may also include supplemental information, such as what, when, and how much of a certain medication was taken, type of food consumption, the weight of the person, the body composition of the subject and so on, which can be entered into the processor 90 using any suitable means. The health-monitoring device may display the insulin sensitivity, as shown in FIG. 1b, or the progression of the insulin resistance, as shown in FIG. 2.

In individuals with normal glycemic values, such as in obesitas or onset metabolic syndrome, the system and method of the present invention may measure the variable FFA levels only. The system may determine the glucose levels by assuming the glucose level to be in the normal range. For example, FIG. 1b illustrates the evolution of the insulin sensitivity factor in the patient over the course of months.

In one embodiment, the health-monitoring device 10 of the present invention may utilize FFA levels for monitoring therapeutic intervention and evaluating the success of therapeutic intervention. Reducing body fat through diet and exercise will reduce early morning and base level FFA and restores insulin sensitivity. FFA levels may be used to measure and monitor the effect of the therapy and possibly allow for dosing guidance. Although base FFA levels drop when losing body fat (due to less supply), before a meals and fasting FFA levels may temporarily increase due to an increased utilization of body fat. It may therefore be necessary to combine the glucose measurement with the FFA level to interpret the FFA level. In addition, post-prandial FFA levels may be more useful for this assessment since the patient will then not be in the fasting state.

FIG. 2 illustrates the use of the health-monitoring device 10 to monitor a successful therapeutic effect over the course of months. As shown, the display 19 of the device 10 may be used to display a graph 21, which tracks a user's insulin resistance factor by graphing a curve 23 over time. The graph may also display a therapeutic goal 22 (graphed as a zone) which was set for the particular patient. The device 10 may compare the user's actual insulin resistance factor with a set goal to provide feedback and motivation to the user.

The device 10 may also be used to monitor insulin dependent diabetes patients. Insulin dependent diabetes patients are characterized, among other elements, by a shortage or even absence of insulin. Typically, these patients are treated through self-administration of insulin. Insulin, which is injected by the patient himself, comes in different forms: some preparations have a very fast and short action profile and are used typically to clear the carbohydrates from the blood stream after a meal. Other preparations have a long half-life time and are used to supply a patient with a more or less stable base amount of insulin throughout the day and night.

It is the duty of the patient to balance the amount of these two insulin types with the size and composition of his meals, exercise, stress levels, sickness, and sleep and wake cycles. The goal of such a treatment is to achieve near-normal glucose levels. Some patients may use an insulin pump, which delivers continuously a self-selected amount of insulin through a catheter. Self-management is daunting task for the average person with diabetes.

A major challenge in the management of insulin dependent diabetes is to endure the night (the longest period of fasting) with close to normal glucose levels while avoiding hypoglycemia. The lack of food intake over this period makes it difficult not to overdose insulin whilst avoiding hyperglycemia. An additional problem facing insulin dependent diabetics is the long period that needs to be covered without an intervention, such as a glucose test, a meal or insulin injection (since the patient is asleep). Hypoglycemia at night is complicated by the absence of external notice of the problem and of external intervention.

Glucose levels tend to fall in the first half of the night as the evening meal is digested and the glucose absorbed into the muscle and liver. The counter-regulating hormones, especially Growth hormone and glucagon, start to stimulate lipolysis to supply the body with FFAs and glycerol as energetic substrates for metabolism. The substitution of Glucose by FFAs for the energy needs saves the further consumption of glucose by muscle and other tissues, freeing up glucose for oxidation by the neural tissues (brain, nerves) to maintain metabolism. Glycerol will contribute to the neogenesis of glucose. Those two elements will cause the glucose level to increase by early morning. Cortisol levels increase as well before waken up and have a similar hyperglycemic effect.

As a result, night hypoglycemia may not be recognized in the early morning glucose values. However the FFA levels before breakfast may give insight in the level of lipolysis occurring overnight, reflecting the degree of hypoglycemia of the previous night period.

Depending on the relative imbalance between the evening and/or bedtime food intake and the amount of injected insulin, glucose levels in the morning can vary substantially.

Thus, high glucose levels in the morning may result from a relative overdose of insulin the evening before. High FFA levels at wake indicate a hypoglycemia overnight. When coinciding with high glucose levels, this condition should not be treated with a higher insulin dose at bedtime.

A milder form of the counter-regulating hormone action is known as the “dawn-phenomenon”, a condition that occurs when a patient wakes up with a high glucose level and high ketone levels (indicative for the enhanced lipolysis) as a reaction to low overnight glucose levels. Insulin dependent patients tend to require more insulin in the morning to lower their blood glucose than during the course of the day. This reduced insulin sensitivity, caused by the counter-regulating hormones (even in absence of night hypoglycemia) may be assessed by measuring FFA levels together with the glucose level in the morning before breakfast. FFA levels can double at wake in the existence of the Dawn-phenomenon.

The health-monitoring device 10 may also be used to provide assistance in determining insulin dosage, based upon both the glucose levels and the FFA levels in the user. For example, the health-monitoring device 10 may be used to retrospectively assess night hypoglycemia utilizing measured FFA levels. As described, glucose levels alone are not ideal to dose insulin. Glucose readings can be normal to very high in the morning as a consequence of hypoglycemia overnight. This situation is rather caused by an over-dosing of insulin relative to the meal intake in the evening. These patients with high glucose and high FFA in the morning should reduce insulin (or increase caloric intake or change meal composition) in the evening rather than take more insulin, which is the natural reflex. Current practice in self-dosing of insulin lacks the counter-regulating hormone information and works with glucose levels alone. Most often this results in patients taking more insulin the next evening to tackle the hyperglycemia. As a consequence, the following night even more severe hypolycemia and consequential hyperglycemia can be the result. It usually takes several days to get back into control.

For example, FIG. 5 shows the display 19 of the health-monitoring device 10 of FIG. 1 according to one embodiment when the device is used to display early morning test results for the two analytes. As shown, the display 19 of FIG. 5 displays a first analyte measurement, illustrated as the free fatty acid measurement and a second analyte measurement, illustrated as the glucose measurement, in measurement region 41. The device compares the measurements to the target range, shown in target region 42. The illustrative device 10 may identify the analyte pattern typical for night hypoglycemia and may provide a diagnosis to the user, shown in diagnosis region 43 of the display 19. For example, as shown, the device 10 may conclude that the patient should reduce his evening insulin to avoid repetition of a night hypoglycemia, as show by the recommendation 45. In addition, as a consequence of the high FFA in the morning, the device may calculate and inform the patient that, for example, 50% more insulin will be needed to tackle the increased insulin resistance, as shown in dosage region 44 of the display 19.

The health monitoring device may also be used to identify over-insulinized patients by measuring FFA levels. The risk exists that the patient becomes trapped in a cycle of increasing his insulin each time he perceives a high glucose reading. Ignorant about the effects of the counter-regulating hormones, a patient may end up with frequent high levels of both FFA and glucose, and a low insulin sensitivity while consuming large amounts of insulin. The medical community has started to recognize this logical self-perpetuating cycle. The only efficient, though intuitively contrary approach is to drastically reduce the insulin intake to restore the hypoglycemia, reduce the FFA levels and improve the insulin sensitivity. Therefore, information regarding FFA combined with glucose levels, as measured and analyzed using the device 10, may provide information early to the patient so he can avoid over-insulinization or restore insulin sensitivity.

As shown in FIG. 1, the device 10 of the present invention may be used to track the evolution of an insulin sensitivity factor in a patient. Depending on the volatility of the insulin sensitivity factor and the therapeutic goals, the time basis for the tracking can be changed, showing the evolution over weeks or days, rather than months. An (averaged) intra-day evolution can reveal even more detailed information. Consistent low insulin sensitivity in the late afternoon, for example, may signal the patient to reduce insulin before lunch or increase the lunch calorie content or composition.

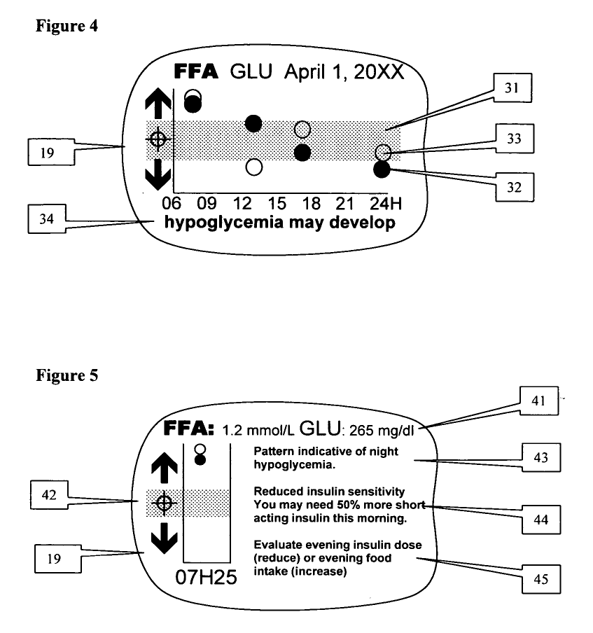

According to another embodiment of the invention, the device 10 can measure and utilize FFA and glucose levels to assess the prospective development of hypoglycemia and hyperglycemia in a patient. For example, FIG. 4 shows use of the device 10 of the illustrative embodiment of the present invention to inform the user regarding potential imminent hypoglycemia, as shown in FIG. 4. The challenge of the night hypoglycemia and “dawn phenomenon” treatment is to avoid low glucose levels overnight primarily by identifying the conditions in advance. FFA levels in combination with glucose measurements, as detected by the device 10, can help to avoid low glucose levels overnight. Certain patterns, such as a normal to low glucose level in the presence of low FFA level at bedtime or early night, may indicate the development of hypoglycemia in the near future. By detecting this pattern, the system and method of the invention may help the patient to take preventive steps to avoid imminent hypoglycemia. The device may provide recommendations to the patient, such as to take an extra snack (with slow absorbing carbohydrates) before going to sleep. As shown in FIG. 4, the device 10 graphs both free fatty acid levels 32 and glucose levels 33 on the display 19 and compares the measured levels to a therapeutic goal, illustrated as region 31. The display may display a message 34 that warns the user of potential hypoglycemia, based on the measured analytes. In FIG. 4, the illustrative device 10 recognizes that although the glucose reading 31 is near normal, the low FFA level 32 indicates a strong insulin activity in spite of the already normal to low glucose level.

In the opposite case, when the glucose level is normal to high in the presence of a high FFA level at bedtime or early night, one might expect a hyperglycemic response. This situation typically occurs when insufficient insulin is available. The high FFA level in combination with the lack of insulin action may result in keto-acidosis, which can be a life threatening condition and may lead to a coma. When such a condition exists, the device 10 can detect the condition and may recommend to the patient to take extra insulin or reduce food intake to correct the condition.

According to another application, the device 10 utilizes FFA and glucose levels to assess the insulin sensitivity and therefore help in determining an appropriate pre-prandial insulin dose. The combined information of glucose and FFA levels in a patient allow the device 10 to assess the insulin sensitivity of the patient. Information regarding a patient's insulin sensitivity can be particularly relevant when the patient has to inject himself with insulin prior to his meal. The insulin is intended to remove glucose from the bloodstream that appears as a result of the meal digestion. When high levels of FFA are present resulting in a low insulin sensitivity, the user must to inject himself with a higher amount of insulin to avoid high glucose levels after the meal. This is particularly helpful before breakfast when FFA levels are usually high.

According to another application, the health-monitoring device may utilize information regarding FFA and glucose levels for closed loop systems and insulin dosing algorithms. Glucose alone as a reflection of carbohydrate metabolism has proven to be insufficient to build reliable dosing algorithms. Systems based on glucose input alone are lacking the essential information from the fat metabolism, counter-regulating hormones and insulin sensitivity. FFA levels combined with glucose, as set forth in the present invention, provide a more complete picture of the actual metabolic state of the patient. The present invention combines the fat and glucose metabolic information as inputs for insulin dosing algorithms. These algorithms may be stand-alone minicomputer or palmtop based systems as well as incorporated in glucose measurement devices or insulin delivery systems (i.e. insulin pen or pump). Some of those algorithms are predictive in such that they assess the expectable glucose levels in the near future.

Closed loop systems are systems that aim to deliver insulin automatically based on inputs from the patient's metabolic state. They consist typically of a measuring or input device for metabolic parameters (i.e., glucose, dietary input, logging exercise and insulin administration), an insulin dosing algorithm and an insulin delivery system.

The present invention has been described relative to an illustrative embodiment. Since certain changes may be made in the above constructions without departing from the scope of the invention, it is intended that all matter contained in the above description or shown in the accompanying drawings be interpreted as illustrative and not in a limiting sense.

It is also to be understood that the following claims are to cover all generic and specific features of the invention described herein, and all statements of the scope of the invention which, as a matter of language, might be said to fall therebetween.

What is claimed is:

1. A method of assessing a user's health comprising the computer implemented steps of:
measuring an amount of a first analyte in a biological fluid sample reflecting body fat metabolism and an amount of a second analyte in the biological fluid sample reflecting glucose metabolism, and
assessing the health of the user based on the amount of the first analyte and the amount of the second analyte.

2. The method of claim 1, further comprising the step of formulating advice to the user based on the step of assessing.

3. The method of claim 2, further comprising the step of using the formulated advice to drive a closed loop system for insulin dosage.

4. The method of claim 1, further comprising calculating and providing a dose for a medication based on the step of assessing.

5. The method of claim 1, wherein the step of assessing the health comprises assessing a metabolic syndrome, and further comprising the step of calculating a therapeutic intervention for the metabolic syndrome.

6. The method of claim 1, wherein the step of assessing the health of the user comprises calculating an insulin sensitivity factor based on the amount of the first analyte and the amount of the second analyte in the biological fluid sample.

7. The method of claim 1, wherein the first analyte comprises at least one fat metabolism analyte.

8. The method of claims 1 wherein the sample comprises one of blood, a derivate of blood, interstitial fluid, urine, saliva and mixtures thereof.

9. The method of claim 1 wherein the step of assessing comprises predicting the likelihood of the user developing hypoglycemia or hyperglycemia.

10. The method of claim 1, wherein the step of assessing comprises utilizing a third parameter comprising one or more of: body mass Index, gender, body composition, meal intake, insulin delivery, medication and weight to assess the health of the user.

11. The method of claim 2, wherein the advice takes the form of a glucose level prediction for a future period.

12. A health-monitoring device for running the computer implemented steps of claim 1.

13. A health-monitoring device for assessing a user's health, comprising:
a sampling device for providing a biological fluid sample from the user; and
one or more test elements for measuring an amount of a first analyte in the biological fluid sample reflecting body fat metabolism and an amount of a second analyte in the biological fluid sample reflecting glucose metabolism.

14. The device of claim 12, further comprising a program for assessing a parameter indicative of the user's health based on the amount of the first analyte and the amount of the second analyte in the sample.

15. The device of claim 13, wherein the parameter is an insulin sensitivity factor.

16. The device of claim 13, wherein the parameter is hypoglycemia.

17. The device of claim 13, wherein the program formulates a recommendation for therapeutic intervention.

18. The device of claim 16, wherein the therapeutic intervention comprises one of:
a dose of insulin, diabetic medication and dietary advice.

19. The device of claim 12, where the test elements comprise one or more of a photometric, reflectometric, electrochemical and fluorescense based system for analyzing the biological fluid sample.

20. The device of claim 12 where a single test element is able measuring both an amount of a first analyte in the biological fluid sample reflecting body fat metabolism and an amount of a second analyte in the biological fluid sample reflecting glucose metabolism.

21. The device of claim 12, wherein the device automatically or continuously measures the second analyte.

22. The device of claim 12, wherein the device automatically or continuously measures the first analyte.

23. The device of claim 12, further comprising a memory element for tracking the evolution of a disease or therapeutic success.

24. A method for monitoring the health of a user, comprising the steps of:
measuring a first analyte in a biological fluid sample reflecting body fat metabolism;
determining a glucose level in a biological fluid sample; and
calculating and tracking the evolution of an insulin resistance factor in the user based on the measured level of the first analyte and the glucose level.

25. The method of claim 22, further comprising the step of predicting the likelihood of a developing hypoglycemia or hyperglycemia.

26. The method of claim 22, wherein the step of calculating and tracking utilizes one or more additional parameters comprising one of: body mass Index, gender, body composition, meal intake, insulin intake, medication and weight.

27. The method of claim 22, wherein the glucose level is determined by measuring an amount of glucose in a biological fluid sample.

28. The method of claim 22, wherein the glucose level is determined by assuming the glucose level to be a normal level.

29. A method of monitoring a health parameter in a user, comprising the computer implemented steps of:
measuring an amount of a fat metabolism analyte in a biological fluid sample reflecting body fat metabolism
measuring an amount of a glucose metabolism analyte in the biological fluid sample, and
correlating the amount of the fat metabolism analyte and the amount of the glucose metabolism analyte to the health parameter.
